(19) United States
(12) Patent Application Publication
(10) Pub. No.: US 2025/0174367 A1
(43) Pub. Date: May 29, 2025

(54) CONNECTIVITY INFRASTRUCTURE FOR A TELEHEALTH PLATFORM WITH THIRD-PARTY WEB SERVICES

(71) Applicant: TELADOC HEALTH, INC., Purchase, NY (US)

(72) Inventors: Parixit Kaira, Santa Barbara, CA (US); John Cody Herzog, Santa Barbara, CA (US); Blair Whitney, Santa Barbara, CA (US)

(21) Appl. No.: 19/041,214

(22) Filed: Jan. 30, 2025

Related U.S. Application Data
(63) Continuation of application No. 16/803,800, filed on Feb. 27, 2020, now abandoned.
Application No. 16/803,800 is a continuation-in-part of application No. 16/114,091, filed on Aug. 27, 2018, now Pat. No. 11,636,944.
(60) Provisional application No. 62/550,514, filed on Aug. 25, 2017.

(51) Int. Cl. G16H 80/00; G16H 15/00; H04N 7/15

(52) U.S. Cl. CPC G16H 80/00; G16H 15/00; H04N 7/152

(57) ABSTRACT

A secure, reliable telehealth delivery platform that also provides flexibility and scalability. The platform includes a plurality of geographically dispersed communication servers that facilitate communication sessions between remotely located patients and healthcare providers over a public communications network. The platform includes a connectivity server that manages access among users and locations. The platform also includes a monitoring server that monitors the health and usage of devices coupled to the network and proactively identifies issues requiring intervention before service interruptions occur. That platform may also provide clients in heavily-restricted network environments with seamless access to multiple third-party web service providers.

### CROSS-REFERENCE TO RELATED APPLICATIONS

This application is a continuation of U.S. application Ser. No. 16/803,800, filed Feb. 27, 2020, which is a continuation-in-part of U.S. application Ser. No. 16/114,091, filed Aug. 27, 2018, which claims priority to U.S. provisional application No. 62/550,514, filed Aug. 25, 2017, the contents of which are hereby incorporated by reference.

### TECHNICAL FIELD

The present technology pertains to telehealth systems, and more specifically to a network and connectivity infrastructure for a telehealth platform.

### BACKGROUND

Telehealth or telemedicine is the practice of conducting consultations between patients and caregivers in different locations. Telemedicine technologies allow physicians and other care providers to see, hear, communicate with, diagnose, instruct or otherwise provide care to remotely located patients. Existing telemedicine technologies range from simple audio communication devices such as telephones to videoconferencing systems to remotely controlled mobile videoconferencing devices with autonomous navigation capabilities. One such remotely controlled device is that marketed under the trademark RP-VITA or INTOUCH VITA by INTOUCH TECHNOLOGIES, INC. of Goleta, California. Other telemedicine devices or “endpoints” offered by INTOUCH TECHNOLOGIES, INC., include the cart-based INTOUCH LITE, INTOUCH VICI, and INTOUCH VANTAGE, the portable INTOUCH EXPRESS and INTOUCH VIEWPOINT or VIEWPOINT TABLET, and the ultra-portable INTOUCH TV, which can be coupled to any television to create a telemedicine endpoint. In addition, any computing device running INTOUCH VIEWPOINT software or a web browser directed to the INTOUCH cloud-based CONSUMER portal can be used to conduct a telemedicine session.

Each of these devices, which are typically situated in the vicinity of a patient, includes at least one camera, monitor, microphone, and speaker that enable two-way audiovisual communication between the patient and the remote physician. In conjunction with a managed network, cloud-based documentation platform, and a purpose-built user interface provided to the physician via a laptop, desktop, or smartphone, these devices allow physicians to communicate with, examine, and diagnose even high-acuity patients from a remote location, as well as retrieve, edit, and store patient medical records and medical imaging from disparate healthcare facility IT systems and electronic health record systems.

Many organizations that wish to offer telehealth services struggle to establish a delivery platform that is both secure and reliable but also flexible and scalable. Complex webs of Virtual Private Networks (VPNs) may provide secure communications among care locations within a healthcare organization, but VPNs tend to suffer from reliability issues that may prevent or otherwise interrupt telehealth sessions at critical times. Moreover, pre-established VPNs generally do not permit sessions with entities outside of the organization that administers the VPN. This may prevent or hinder the ability of an external specialist to join and contribute to a session where his or her expertise may be helpful.

### SUMMARY OF THE CLAIMED SUBJECT MATTER

One aspect of the disclosure is a telehealth system comprising a public communications network (PCN), a first server coupled to the PCN, a second server coupled to the PCN, and a user device coupled to the PCN via a firewall. The first server has a first address on the PCN. The second server has a second address on the PCN. The firewall is configured to allow communications between the user device and the first address and block communications between the user device and the second address. When the first server receives a request from the user device that includes a request for a service provided by the second server, the first server relays the request from the user device to the second server and relays a response to the request from the second server to the user device.

Another aspect of the disclosure is a method in a telehealth server. The method includes receiving a first request from a client device via a firewall. The firewall is configured to allow connections between the client device and the telehealth server and block connections between the client device and a second server. The first request includes a first portion that identifies the telehealth server and a second portion that identifies a target resource. The method further includes generating a second request for the target resource and transmitting the second request to the second server. Finally, the method includes receiving a response to the second request from the second server and transmitting the response to the client device via the firewall.

### BRIEF SUMMARY OF THE DRAWINGS

FIG. 1 illustrates an exemplary telehealth platform.

FIG. 2 illustrates an exemplary interactive access map.

FIG. 3 illustrates an exemplary telehealth platform configured to provide multiple web services.

### DETAILED DESCRIPTION

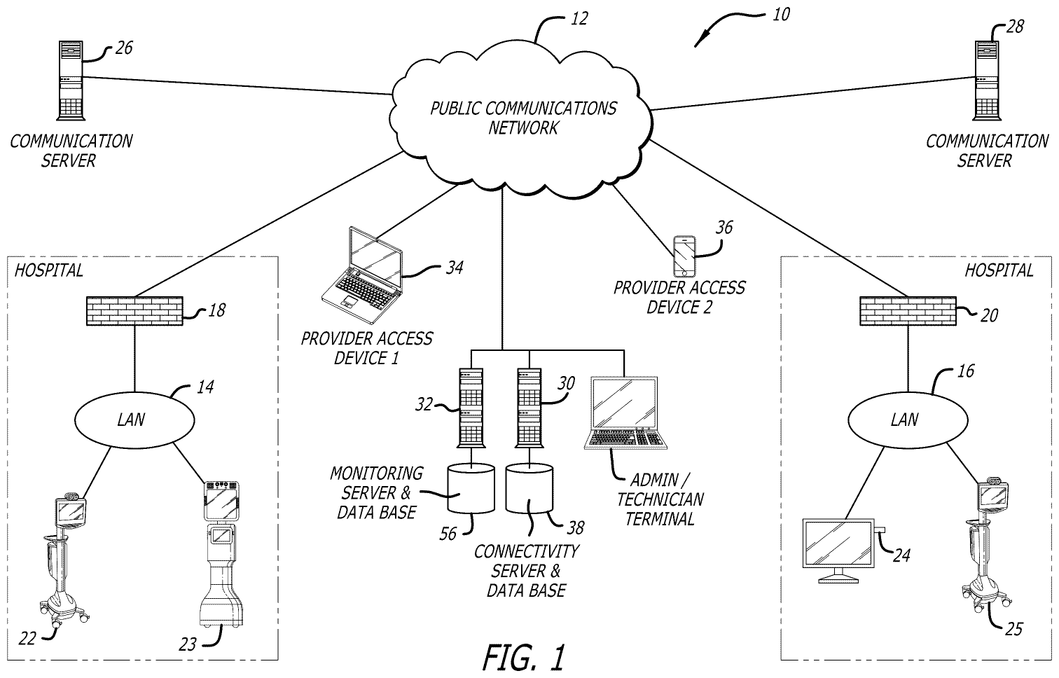

FIG. 1 illustrates an example of a telehealth platform. The platform may include a public communications network (PCN), such as the Internet, that connects numerous local-area networks (LANs). Each LAN may be coupled to the PCN via a communications firewall. One or more patient access devices may be coupled to each LAN. A patient access device may also be coupled to the PCN via a wide-area network (WAN), such as a cellular communications network. The platform may include a plurality of communications servers, a connectivity server, and a monitoring server coupled to the PCN. The platform may also include one or more provider access devices coupled to the PCN via a LAN or WAN. In general, the platform allows a physician or other healthcare provider to provide care to a patient by establishing a two-way audio/video communication between a patient access device and a provider access device. Although patient access devices and provider access devices may be discussed as distinct and/or purpose-built devices, it is to be appreciated that the platform accommodates connections between generic users and/or devices.

Each LAN may be associated with a medical facility, such as a hospital, clinic, surgery center, primary care facility, ambulatory care center, or nursing home. A LAN could also be associated with a patient's home and/or the home or office of a care provider, such as a physician, clinician, nurse, or caretaker. For larger organizations, a LAN could take the form of a wide-area network (WAN) that connects multiple LANs. The LAN or WAN associated with each medical facility may also employ or otherwise support one or more virtual private networks (VPNs). However, the managed network architecture of the platform generally eliminates the need for VPNs.

Each LAN may be coupled to the public communications network via a firewall. Each firewall may be configured by an administrator to allow incoming connections from one or more IP addresses representing the IP addresses of one or more of the communication servers, the monitoring server, and/or the connectivity server. For example, the administrator may be provided with a list of trusted IP addresses and/or port numbers associated with the telehealth platform and configure the firewall to accept connections from these IP addresses and/or ports.

An exemplary patient access device may take the form of any device that allows the user of the provider access device to see and hear the patient. Preferably, the patient access device also allows the patient to see and hear the provider. In one embodiment, a patient access device may take the form of a telemedicine cart including a camera, a monitor, a microphone, and a speaker. In another embodiment, a patient access device could be a mobile telepresence robot that includes a camera, a monitor, a microphone, and a speaker, as well as a mobile base that can be controlled by the remote healthcare provider using the provider access device. In yet another embodiment, the patient access device could take the form of a small form factor computing device that executes a patient access device software program and is coupled to a camera, a monitor, a microphone, and a speaker via input/output ports on the computing device. One such small form factor computing device is the COMPUTE STICK, manufactured by INTEL Corporation. Alternatively, the patient access device may take the form of videoconferencing enabled tablet, smartphone, or laptop or desktop computer executing a patient access device software program. Additional examples of patient access devices include INTOUCH VITA, INTOUCH LITE, INTOUCH VANTAGE, INTOUCH XPRESS, INTOUCH VICI, and INTOUCH TV, all marketed by INTOUCH TECHNOLOGIES, INC., of Goleta, California. The details of various examples of provider access devices may be found in the follow U.S. patent and application publication numbers, the contents of which are hereby incorporated by reference: U.S. Pat. Nos. 6,925,357; 7,158,859; 8,515,577; 8,670,017; 8,718,837; 8,996,165; 9,198,728; 2009/0240371; and 2011/0213210.

An exemplary provider access device may take the form of any laptop or desktop computer, tablet, smartphone, or videoconferencing device including a camera, a monitor, a microphone, and a speaker. The provider access device may include a provider access software program that, when launched, provides an integrated application and user interface for accessing, controlling, or otherwise communicating with patient access devices in order to treat a patient remotely. In addition, the provider access software may include the ability to launch a clinical documentation tool the physician can use to document the patient encounter, either within the provider access interface or within a separate application window launched from within the provider access software interface. The clinical documentation tool may be integrated with a hospital medical records system. The provider access software may also allow the provider to retrieve and view medical images of the patient, such as PACS images from MRIs or CT scans that have been performed on the patient. The provider access software may allow the provider to view these images within the application window of the provider access software or within a separate application window launched from within the provider access software interface.

In an alternative embodiment, the provider access device may simply employ a web browser that the user directs to a URL having a web-based provider access software application. This embodiment avoids the need to have a native application installed on the provider access device.

In yet another embodiment, both platform may include a cloud-based telehealth portal configured to host telehealth sessions between any videoconferencing enabled devices via web browsers directed to and authenticated with the telehealth portal.

In general, the provider access software interface may include a variety of layouts and features as described in the following U.S. patent and application publication numbers, the contents of which are hereby incorporated by reference: U.S. Pat. Nos. 8,179,418; 8,849,680; 9,361,021; 9,098,611; 2005/0204438; and 2006/0052676. In addition, the provider access software interface may be customized depending on the type of patient access device the user chooses to connect to, as described in U.S. Pat. No. 8,897,920, the contents of which are hereby incorporated by reference.

The platform may include one or more geographically dispersed communications servers coupled to the public communications network. An exemplary communication server may operate as a session initiation protocol (SIP) server or similar protocol for signaling and controlling multimedia communication session over the internet. Each of the communications server is configured to facilitate a multimedia communication sessions between a provide access device and a patient access device and/or to facilitate communication between a patient or provider access device and other servers coupled to the PCN, or between other servers themselves. The other servers could include another communications server, the monitoring server, the connectivity server, an authentication server, a medical records server, a billing server, a reporting server, or the like. In a platform with multiple, geographically dispersed communications servers, an optimal server to facilitate a session between a provider access device and a patient access device may be selected based on server load and/or network conditions such as latency, available link bandwidth, and/or round-trip time.

The connectivity server maintains a database of connectivity rules that control access throughout the platform. Each connectivity rule may specify a user and a location. A user may correspond to a medical care provider. A location in this context is a unique identifier that may represent the name of the location of a specific patient access device. For example, if the device is the only patient access device located in the emergency department of Mercy General Hospital in Asheville, NC, the location may be named “Mercy General, Ashville, ED.” A connectivity rule may indicate that a “Dr. Jim Norris” can connect to or otherwise access the patient access device located at “Mercy General, Ashville, ED.” The location may be distinct and/or decoupled from the name of the hospital or facility, the customer, the physical location of the device (in terms of latitude and longitude), and/or the device ID or serial number.

The database of connectivity rules may be maintained by an administrator via a web portal that allows the administrator to add, delete, or otherwise modify connectivity rules. For example, when a new doctor is registered and obtains an account on the telehealth platform, one or more connectivity rules may be added to the database, with each rule specifying the doctor's username and a patient access device location he or she is authorized to access. A single provider may have access to numerous patient access devices located throughout one or more hospitals, hospital networks, or other care locations. In such cases, the database may include a separate rule for each patient access device location the provider is authorized to access. In other embodiments, a single connectivity rule for a provider could specify a group of patient access devices the provider is authorized to access. A provider may not be able to connect to a patient access device location if there is no corresponding connectivity rule associating the provider with the patient access device.

In one embodiment, connectivity between users and locations may be grouped or otherwise managed according to “programs” maintained in the connectivity database. A program may be a unique name representing a set of users that have access to the devices at a set of locations for a particular purpose. For example, a major hospital with several neurologists on staff may contract with several smaller hospitals in remote locations to provide stroke tele-consults at the remote locations. Once telehealth endpoints are installed or otherwise made available at the remote hospitals, a program may be created in the database that includes the neurologists at the major hospital in the users list and the locations of the telehealth devices at each of the remote hospitals in the locations list. This grouping may automatically create rules in the connectivity server that allow each user in the users list to have access to each location in the locations list.

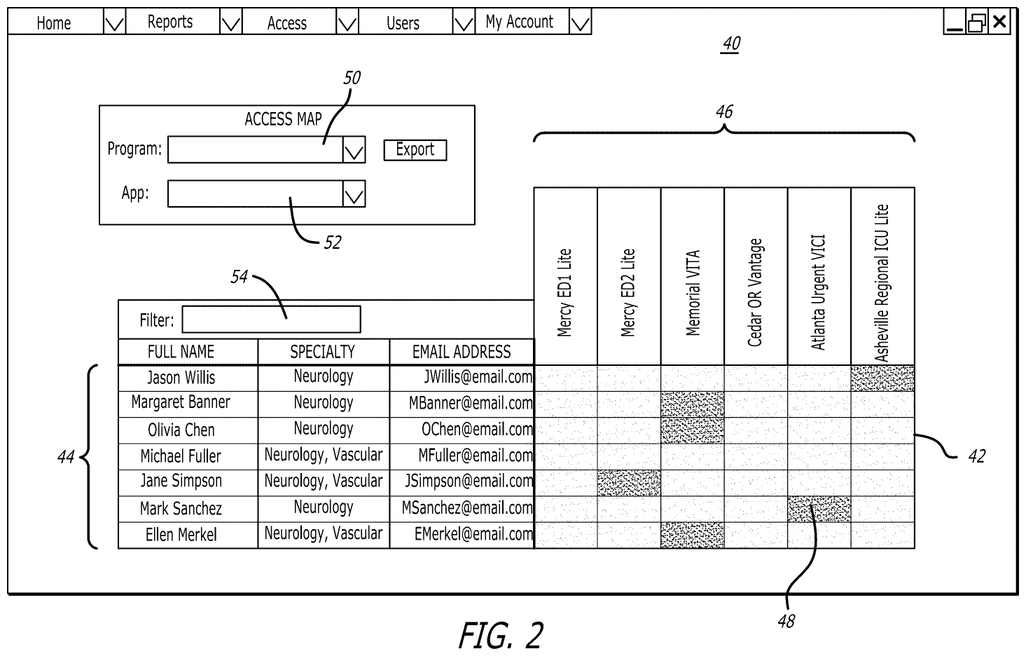

The connectivity server may provide an graphical user interface that includes an interactive access map for each program. FIG. 2 shows an example of an interactive access map for a stroke program associated with a Mercy General hospital. The map includes a vertical list of the names and specialties of users on the left that have been added to the users list of the Mercy General stroke program. The map also includes a horizontal list of locations that have been added to the locations list of the Mercy General stroke program. By default, every user in the users list of a program may have access to every location in the locations list of that program. However, in one embodiment, the access map may allow access between specific users and locations to be toggled. For example, as shown in FIG. 2, connectivity is enabled for all of the tiles (each representing a combination of a user and a location) filled gray. However, connectivity has been disabled at one location for each user, as illustrated by the tiles filled black. An administrator may toggle connectivity for any user-location combination by clicking on the corresponding tile. The system may also allow the administrator to toggle the connectivity of several users and locations at once using a “CTRL-click” input on various tiles or dragging and highlighting a group of contiguous tiles using a mouse of keyboard input.

The administrator may select different access maps by selecting different programs from the drop-down menu labelled “Program” in the upper-left corner of the interface. The interface may also include another drop-down menu labelled “App” in the upper-left corner that allows the administrator to view access among users and locations across different applications, such as access to remote presence (e.g., two-way audiovisual sessions), access to clinical documentation tools, and/or access to medical records or imagery. The access map may additionally include a filter function to allow the administrator to filter the list of user or locations to find specific users or locations more easily.

The monitoring server maintains a database of status information from each of the patient access devices. The status information may be reported to the monitoring server from the patient access devices periodically, in response to polling by the monitoring server, or in response to an event. Exemplary status information may include detailed device status information, including battery life, LAN ID and connection status, wireless signal strength, whether the device is coupled to a charging station and/or AC power, software version, date and time of last reboot, and other device-related information. The monitoring server also maintains a record of all communications sessions between a patient access device and a provider access device, including the patient access device location and/or device ID, the provider username or ID, the duration of the session, the quality of the session, and other session-related information.

All or a portion of the database of connectivity rules maintained in the connectivity server is mirrored in one or more of the communication servers. When a user or care provider wishes to connect to a patient access device to conduct a session with a patient, he or she launches a provider access software program on his or her provider access device. In another embodiment, the provider may simply direct a web browser to a web-based provider access interface. The user supplies login credentials, such as a username and password, which are transmitted to the communications server for authentication, or to a separate authentication server in communication with the communications server. Once the user is authenticated, the communications server sends to the provider access device a list of patient access devices to which the provider is permitted to connect, as well as a status of each of the devices at the corresponding locations. The device statuses may include ready, busy, and/or offline, depending on whether the device is online and available to participate in a communication session, online but in use by another provider, or offline, respectively.

From the displayed list of patient access devices the provider may then select an available patient access device to connect to, after which the provider access device transmits a request to connect to the patient access device to the communications server. The communications server then begins a process to the establish a communication session between the provider access device and the patient access device. The communications server may employ a process such as that described in U.S. Pat. No. 9,160,783, the contents of which are hereby incorporated by reference. In general, the communication server may first attempt to establish a peer-to-peer session between the patient and provider access devices using firewall traversal techniques such as UDP hole punching and/or those described in ICE/STUN/TURN protocols. If a peer-to-peer session cannot be established between the patient and provider access devices, the server may then attempt to establish a session using itself or another TURN server as a media relay server that relays media back and forth between the patient and provider access devices. In addition, the patient and provider access devices may employ a dynamic bandwidth allocation scheme in order to maximize the quality of the audio/video session through changing network conditions. The dynamic bandwidth allocation scheme may include that described in U.S. Pat. No. 9,264,664, the contents of which are hereby incorporated by reference.

The details of each communication session are logged in the monitoring server and made available to a reporting server. The details of each session may include a username or other identification of the provider, the patient access device location, a serial number or identification of the patient access device, the type of patient access device, the duration of the session, whether the provider accessed the patient's medical records, and/or whether the provider accessed the patient's medical imagery. The identification of the provider may itself be associated with additional details about the provider, such as whether the provider is a part of one or more groups of providers or other healthcare organizations, the name of any such group of providers, and/or the provider's medical specialty or specialties. Similarly, each patient access device location may be associated with additional details regarding the patient access device location, such as the owner, custodian, and/or customer associated with the patient access device location, whether the location is associated with one or more hospitals or healthcare organizations, the name of any such the healthcare organizations, the physical location of the patient access device location, one or more service lines or medical specialties the patient access device is assigned to.

The reporting server may be configured to join usage data of providers and/or patient access device locations with details about each to generate interactive dashboards and reports for visualizing various aspects of the utilization of the telehealth platform. Details of this utilization reporting can be found in U.S. Pat. Nos. 8,902,278 and 9,251,313, the contents of which are hereby incorporated by reference.

The monitoring server may be configured to generate reports including patient access devices and their associated device health information for review by a technician at a remote terminal coupled to the PCN. The monitoring server may additionally be configured to analyze the device health data received form the patient access devices and detect an abnormal condition in one or more of the patient access devices. The analysis of the device health data may include artificial intelligence techniques such as unsupervised or supervised machine learning for anomaly detection. When the monitoring server detects an abnormal condition, the monitoring server may generate an alert to a technician identifying the patient access device requiring attention. Alternatively, the monitoring server may attempt to alleviate the abnormal condition by automatically performing a corrective action. In one embodiment, before generating an alert for a technician, the monitoring server may trigger a reboot for a patient access device that has been identified as having an abnormal condition. In some cases, rebooting the patient access device may correct the abnormal condition and alleviate the need for a technician to service the device.

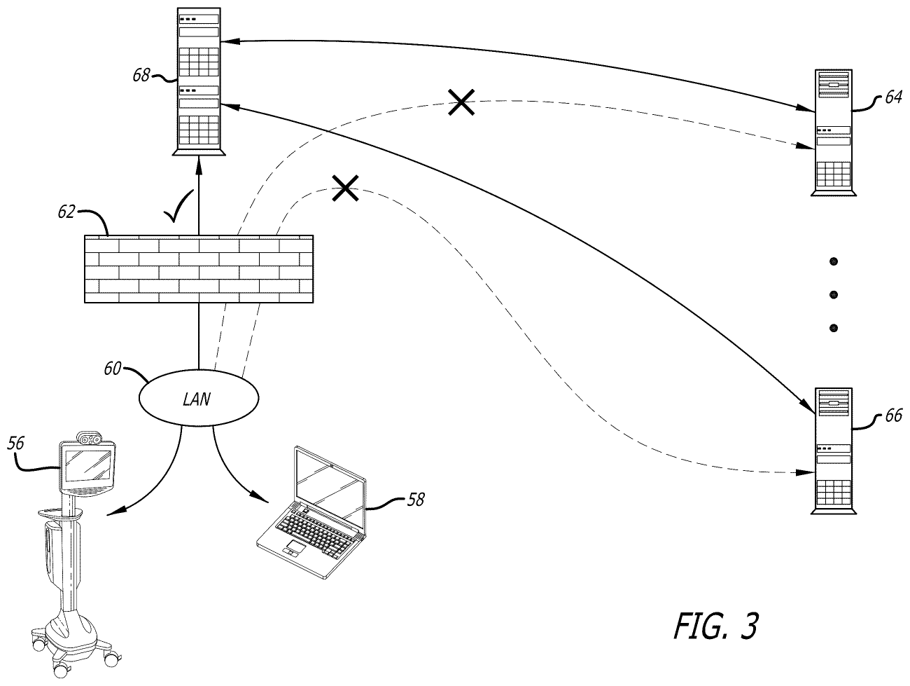

FIG. 3 illustrates an embodiment of the disclosed telehealth platform that facilitates use of multiple, disparate, third-party web services provided by servers owned and/or operated by third-party vendors. Such services are sometimes referred to as “cloud services” or “Software-as-a-Service” (“SaaS”). These services may include necessary or desirable components of telehealth sessions conducted using the client software. In one example, a single telehealth session may involve a number of services that may be provided by different servers and/or web-based service providers: audiovisual conferencing; clinical documentation; web-based pharmacy portals for ordering prescriptions; medical imaging; user authentication; access control; support/live assistance; live language translation services; logging and/or analytics; various networking protocols such as TCP, UDP, ICE, STUN, TURN, and/or persistent web sockets; and/or any other web or cloud-based service.

Many hospitals and other healthcare facilities have heavily restricted communication networks that only permit connections with trusted IP addresses that have been previously vetted with the facility's information technology teams. Thus, in past, enabling a telehealth session with a variety of different web services would has required each facility's IT team to audit each service individually and open their Internet firewalls to every IP address required by each of the various services (a process sometimes referred to as “whitelisting”). In addition, each whitelisted IP address creates an additional burden on the hospital IT staff to periodically re-audit the service and ensure that any whitelisted IP addresses are still be used by the service. Moreover, any time the hospital wishes to switch a to different provider, or the service provider wishes to move the service to a new IP address, the whitelisting process must be repeated before the service can be made available again.

The disclosed telehealth platform resolves these issues by redirecting all communications between the user device and third-party webservices through one or more of the previously whitelisted servers associated with the telehealth platform.

FIG. 3 illustrates an exemplary scenario in which one or more endpoints 56 or user devices 58 on a hospital network 60 behind a firewall 62 needs to communicate with one or more third-party web service providers 64, 66. As illustrated by the broken lines in FIG. 3, the hospital's firewall 62 configuration prevents the devices 56, 58 from establishing a connection with the service providers 64, 66. However, client software on the devices 56, 58 may be configured to override or intercept a target URL—i.e., any call, request, or communication directed to a URL of one of the third-party service providers—with a new or custom URL that refers to one of the whitelisted telehealth servers 68 associated with the telehealth platform. The telehealth server 68 may be one of the communications servers 26, 28 discussed above, a load balancing server, which may be disposed between the hospital network and the communication servers 26, 28, or any other whitelisted server associated with the telehealth platform 10.

The custom URL may include a first portion that includes the domain name, host name, or other name and/or path of the telehealth server 68 and a second portion that is associated with the target URL. The second portion of the customer URL may take the form of an extension, suffix, or other argument of the first portion of the customer URL and is associated with the target URL of the requested third-party service provider. Upon receiving the request with the custom URL, the telehealth server 68 uses the second portion of the customer URL to recreate the target URL and transmits the request to the target URL. Responses to the target URL request from the third-party service provider received by the telehealth server are then transmitted by the telehealth server back through the hospital firewall to the requesting device. In this way, the devices 56, 58 behind the hospital firewall may access the desired third-party service providers 64, 66.

The client software on devices 56, 58 may be configured to override or intercept web requests for third-party service providers from any libraries, APIs, or third-party service provider modules embedded or called from within the client software. In one embodiment, the client software may include one or more wrappers for any third-party service provider software modules configured to intercept any web requests including target URLs directed to third-party web services. These intercepted requests may be replaced with requests to custom URLs that are directed to the telehealth server 68. By way of example, some combination or derivative of the host name, domain name, and/or path of any third-party service provider URLs associated with these requests may be appended to the path of a custom URL with the host name of the telehealth server 68. In general, target URLs or web requests directed to third-party service providers will be transformed into custom URLs that include a first portion, such as the host name, that corresponds to the telehealth server 68, and a second portion, such as a path or any other argument or suffix of the custom URL, associated with the target resource of the third-party service provider. By way of example, the client software may intercept, override, or otherwise replace the following target URL,
https://static.vidconfsaas.com/v3.39/js/vidconfsaas.min.js
   with the following custom URL,
https://saas.telehealthserver.com/vidconfsaas/v3.39/js/vidconfsaas.min.js
   Because the telehealth server 68 is whitelisted, this request is passed through the firewall 62 to the telehealth server 68.

The telehealth server 68 may be configured to forward each request received from the client software to the target URL or, more generally, the address of a server or other device associated with the third-party service provider 64, 66 corresponding to the request. Responses from the third party service providers may be routed back to the telehealth server 68, which may then forward the response back through the hospital firewall 62 and on to the originating or intended device 56, 58. The forwarding function of the telehealth server 68 may be configurable from another device, such as the admin terminal illustrated in FIG. 1 or any other device coupled to the PCM 12 in FIG. 1. By way of example, an administrator of the telehealth platform may maintain a forwarding table that is pushed to or otherwise mirrored on the telehealth server 68 that associates each custom URL received from the client software with the target URL of the target resource of the third-party service provider. When the telehealth server 68 receives the custom URL from the client software, it converts the custom URL to the target URL of the third-party service provider and transmits the request to the third-party service provider. The conversion from custom URL to target URL may be accomplished using a look-up table, a function or routine that algorithmically rewrites the custom URL to obtain the target URL, or any other method suitable for mapping the custom URL to the target URL.

This configuration provides several benefits. First, the number of IP addresses that must be whitelisted in the hospital firewall is minimized. This not only simplifies network administration but also improves network security by minimizing avenues of attack from potential hackers. Second, the telehealth platform administrator can seamlessly change servers and/or vendors for a particular SaaS service without requiring action by, or even notification of, hospital IT administrators. This further reduces the administrative burden on hospital IT administrators. For example, the telehealth platform administrator may switch from one videoconferencing SaaS provider to another videoconferencing SaaS provider by merely updating the forwarding table without requiring any action on the part of the hospital IT team. This operation would not be possible if the devices 56, 58 behind the hospital firewall 62 attempted to connect directly to third-party service providers 64, 66. In addition, this configuration allows the telehealth platform administrator to control access to each third party service provider for each user of the telehealth platform. For example, the telehealth platform administrator may create access rules that allow users associated with a first hospital to access a particular third-party service provider while denying users associated with a second hospital access to that third-party service provider. Finally, this configuration allows the telehealth platform administrator to server a single point of contact with the hospital administrator for all third-party web services.

Although the various servers such as the communications servers, monitoring server, connectivity server, and their associated databases may be illustrated or described as being separate devices, it is to be appreciated that any combination of their respective functionalities could be combined or hosted on a single or any number of physical devices coupled to the public communications network.

Additionally, as will be appreciated by one of ordinary skill in the art, principles of the present disclosure may be reflected in a computer program product on a computer-readable storage medium having computer-readable program code embodied in the storage medium, the computer-readable program code executable by a processor. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

While certain exemplary embodiments have been described and shown in the accompanying drawings, it is to be understood that such embodiments are merely illustrative and not restrictive on the broader disclosure, and that this disclosure not be limited to the specific constructions and arrangements shown and described, since various other modifications may occur to those ordinarily skilled in the art.

What is claimed is:

1. A telehealth system that provides access to a third-web service behind a firewall, comprising:
a public communications network (PCN);
a first server coupled to the PCN, the first server having a first address on the PCN;
a second server coupled to the PCN, the second server having a second address on the PCN;
a user device coupled to the PCN via a firewall that is configured to allow communications between the first address and the user device and block communications between the second address and the user device, the user device includes a processor that executes a client software configured to:
receive a user request for a service provided by the second server;
generate a custom uniform resource locator (URL) that includes an alphanumeric string containing a first URL that identifies the first server and a second URL that identifies a service provided by the second server,
wherein, when the first server receives the custom URL from the user device, transmits the second URL to the second server, and transmits a response received from the second server to the user device, and wherein the user device displays content associated with the service provided by the second device.

2. The system of claim 1, further comprising an access control server that can be configured to deny access by the user device to the service provided by the second server.

3. The system of claim 2, wherein the service is a videoconferencing service.

4. The system of claim 3, wherein the videoconferencing service receives video from the user device via the first server and transmits the video from the user device to a second user device coupled to the PCN.

5. The system of claim 3, further comprising a third server that provides a clinical documentation service accessible to the user device via the first server.

6. The system of claim 4, wherein the access control server can be configured to deny access by the user device to the clinical documentation service.

7. The system of claim 1, further comprising a third server, wherein the first server relays a first request for the service provided by the second server to the second server and relays a second request for the service to the third server.

8. A method for providing access to a third-party web service behind a firewall in a telehealth system, the method comprising:
receiving, at a client device, a user request for a service provided by a third-party server via a firewall configured to allow connections between the client device and a telehealth server and block connections between the client device and the third-party server;
generating a custom uniform resource locator (URL) that includes an alphanumeric string containing a first URL that identifies the telehealth server and a second URL that identifies the requested service,
wherein, the telehealth server receives the custom URL from the user device, transmits the second URL to the third-party server, and transmits a response received from the third-party server to the user device via the firewall.

9. The method of claim 8, wherein generating the custom URL includes determining a host name of the third-party server using the second URL.

10. The method of claim 9, wherein generating the custom URL includes appending a portion of the second URL to the host name of the third-party server.

11. The method of claim 9, further comprising changing the host name of the third-party server to the host name of a third server.

12. The method of claim 11, further comprising receiving a second request from the client device, the second request including a first URL that identifies the telehealth server and a third URL that identifies a second third-party service.

13. The method of claim 12, further comprising generating a second custom URL by appending the a portion of the third URL to a host name of the second third-party server.

14. The method of claim 8, wherein the requested service is a videoconferencing service.

15. The method of claim 14, further comprising:
receiving a second request from the client device, the second request including a first URL that identifies the telehealth server and a second URL that identifies a second service associated with a third server;
generating a second custom URL the second service;
transmitting the second custom URL to the third server;
receiving a response to the second request from the third server; and
transmitting the response to the second request to the client device via the firewall.

16. The method of claim 15, wherein the second target resource is a medical imaging system.
